(19) United States
(12) Patent Application Publication
(10) Pub. No.: US 2019/0047498 A1
(43) Pub. Date: Feb. 14, 2019

(54) ADAPTIVE DISPLAY FOR PREVENTING MOTION SICKNESS

(71) Applicant: Intel Corporation, Santa Clara, CA (US)

(72) Inventors: Joelle Alcaidinho, San Jose, CA (US); Glen J. Anderson, Beaverton, OR (US); Oleg Pogorelik, Lapid (IL); Omar Florez, Sunnyvale, CA (US)

(21) Appl. No.: 15/869,331

(22) Filed: Jan. 12, 2018

(51) Int. Cl. B60R 16/037; G06F 3/01; G06F 3/14; G06F 3/16; B60H 1/00; A61M 21/00

(52) U.S. Cl. CPC B60R 16/037; G06F 3/013; G06F 3/14; G01C 21/18; B60H 1/00742; A61M 21/00; G06F 3/165

(57) ABSTRACT

Systems and techniques for an adaptive display for preventing motion sickness are described herein. In an example, an adaptive display system is adapted to determine, such as from sensor information, movement of a vehicle. The sensor information may be obtained from sensors installed in the vehicle. The adaptive display system may display, to a passenger of the vehicle, visual content that changes orientation in correspondence to the movement of the vehicle. The adaptive display system may be further adapted to determine a gaze direction of the passenger and display the visual content in the gaze direction of the passenger. The adaptive display system may be further adapted to obtain physiological data about the passenger and determine the passenger is experiencing motion sickness.

### TECHNICAL FIELD

Embodiments described herein generally relate to adaptive projections and displays for preventing motion sickness and, in some embodiments, more specifically to an adaptive display and environment usable by humans and animals.

### BACKGROUND

Motion sickness may occur when sensory inputs regarding body position in space are contradictory or are different from those predicted from a person or animal's experience. Motion sickness may result from a mismatch between the body's mechanisms responsible for motion sensing and understanding. The mismatch may occur between the semicircular canals of the inner ear, which are responsible for balance and space orientation, and eyes, which provide visual orientation inputs. Motion sickness may cause vomiting, headaches, sweating, yawning, increased saliva, pallor, nausea, and other physical disorders.

Some pharmacological countermeasures to prevent motion sickness have proven effective, but drugs may have significant side effects and latency for effectiveness after taken. Some recommended motion sickness countermeasures are behavioral. Behavioral countermeasures may include having a stable external horizon reference, reducing head movements, sitting in the front seat, and aligning the head and the body with gravito-inertial force. However, young children and animals, such as dogs, may not have the ability to make behavioral countermeasures, such altering their location in the car or focusing on the horizon. Thus, young children and animals may be limited to the use of an undesirable pharmacological solution to avoid motion sickness.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example process for adapting a display for compensating for motion sickness, according to an embodiment.

FIG. 2 illustrates an example of an adaptive display for preventing motion sickness, according to an embodiment.

FIG. 3 illustrates the adaptive display system engine in accordance with some embodiments.

FIG. 4 illustrates a flow diagram of an example of a process for adaptive display for preventing motion sickness, according to an embodiment.

FIG. 5 illustrates the directions of movement a vehicle may experience, according to an embodiment.

FIGS. 6A-6D illustrate examples of the motion forces a person may experience as a passenger in a vehicle in accordance with an embodiment.

FIG. 7 illustrates a flowchart showing a technique for adapting a display for motion sickness in a vehicle in accordance with some embodiments.

FIG. 8 is a block diagram illustrating an example of a machine upon which one or more embodiments may be implemented.

### DETAILED DESCRIPTION

Passengers in a vehicle, such as an automobile, may experience motion sickness. Current remedies for motion sickness include behavioral practices or pharmaceuticals. Because of their position in the vehicle, young children and animals are two of the most motion sickness susceptible passengers as they may not see out the windows. But pharmaceuticals may have undesirable side effects for types of passengers. Additionally, young children and animals are not capable of making behavioral changes to counter the effects of motion sickness.

The presently disclosed system may reduce motion sickness in people and animals by displaying or projecting images and color palettes. In an example, such images and color palettes are displayed inside a vehicle to combat motion sickness caused by incompatibility between motion perception and visual perception. The image of the displays may move in response to vehicle motion to provide a more perceptually compatible visual field.

In an example, an adaptive display system may render an image to match the motion of the vehicle and the content of the image based on objects of interest for humans or animals. By focusing visual attention to a fixed location may help to alleviate motion sickness. Attention can be more effective when it is posed on objects of interest or familiar for the user or animal. Thus the content of the projection or display may be tailored to objects or programming the user enjoys.

In an embodiment, the adaptive display system may project an image or a video. The projected horizon of the image may not move with the vehicle but would stay consistent with the view outside the window so that as a person's head is moved and swayed by the vehicle movement, the projection would have a movement effect, similar to the person's head. The system may alternate views and correction levels that correspond to the best reactions of the user.

In an example, the adaptive display system may display an image on a screen. For example, the vehicle may be outfitted with an in-vehicle infotainment (IVI) system that includes displays for watching programming such as movies and television shows. The IVI system may include an adaptive display system to manipulate the video output to reflect vehicle acceleration and motions, so that the resulted displayed video may be synchronized with the motions sensed by the passenger in the vehicle. The passenger may have their motion and vision senses coordinated which may eliminate a primary cause of the motion sickness.

A vehicle may be equipped with sensors to determine the motions the vehicle is experiencing. Sensors may include cameras, accelerometers, gyroscopes, and a global positioning system (GPS). The adaptive display system may receive data from the vehicle's sensors to determine the motions of the vehicle and correlate those motions to the motions being experienced by a passenger's body. The adaptive display system may adapt and adjust the display or projection to move in a similar fashion, such that the movement of the display or projection is similar to the motions being experienced by the passenger's body.

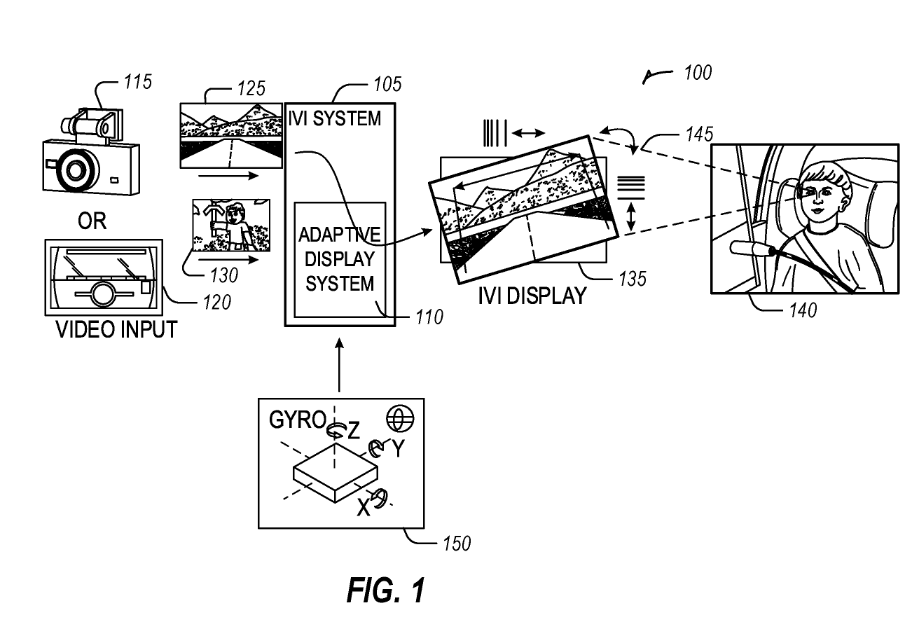

FIG. 1 illustrates an example process 100 for adapting a display for compensating for motion sickness, according to some embodiments. A vehicle may be installed with an IVI system 105, including an adaptive display system 110. The IVI system 105 may receive input from a camera 115 or a video playback device 120. The camera 115 may capture images such as a view of the road 125. The video playback device 120 may play content 130, such as movies, television shows, or video games. The IVI system 105 receives input from sources such as a camera 115 or video playback device 120. The IVI system 105 may transmit the input video to one or more display screens or a projector in the vehicle.

An IVI system 105 may include or be operably coupled to an adaptive display system 110 to modify the video input to provide a passenger 140 with relief for motion sickness. The adaptive display system 110 may receive data about the movement of the vehicle from a sensor such as a gyroscope 150. The adaptive display system 110 may translate the movement data to its effect on a passenger 140. Based on this translation, the adaptive display system 110 may alter the display 135 of the video for the passenger 140. The movements of the altered video on the display 135 may correspond with the motions being sensed by the passenger 140 reduce the effects of motion sickness.

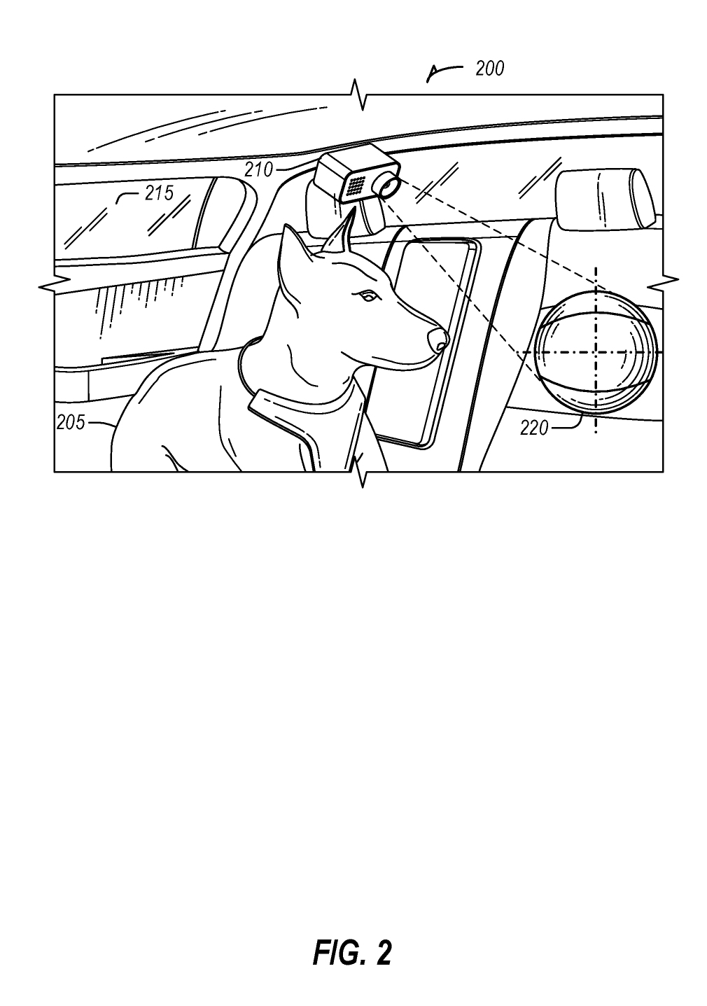

FIG. 2 illustrates an example of an adaptive display for preventing motion sickness 200, according to an embodiment. The example includes a dog 205, which may be riding in the back seat of a vehicle. The dog 205 is not able to see out of the windows 215 of the vehicle. The vehicle, while travelling, experiences various motions, including forward and backward motions from acceleration and deceleration, up and down motions from bumps or dips in the road, and side to side motions when the vehicle turns. When the dog 205 cannot see out of the windows 215, their view is static, such as only seeing the back of the front seats of the vehicle, and does not move in relation to the view of the dog 205. However the dog 205 is sensing the motions of the vehicle as it travels, and thus the motions sensed by the dog 205 and the view of the dog 205 do not correlate. The adaptive display system may include a projector 210 to project an image 220 for the dog 205. The adaptive display system may move the image 220 is relation to the movements of the vehicle so that the motions sensed by the dog 205 and the view of the dog 205 correlate.

The adaptive display system may include a camera to monitor the passengers. The camera may be used to determine the direction of gaze for a passenger, such as a dog 205. Based on the direction of gaze for the passenger, the projector 210 in the vehicle may project an image 220 such that it is within the direction of gaze of the passenger. In an example, the projector may be mechanized with the ability to rotate and adjust the angulation up and down to project an image to different locations within the vehicle. The projector may be attached to a track within the vehicle such that the projector may move along the track to project images to different locations within the vehicle. The rotation, angulation, and track movement may be performed by a motor and controlled automatically by the adaptive display system. This may be beneficial for passengers such as animals and very small children for whom it may not be easy to direct their attention in a specific direction, such as a screen showing a movie. Thus, determining the direction of gaze and using a projector 210 may allow the adaptive display system to position an image in the direction of the gaze of the passenger, to enable the passenger to view the image and be assisted by the adaptive display system without having to consciously look in a specific direction.

In humans, a predominant indication of motion sickness is vomiting, with symptoms also including stomach awareness, sweating, facial pallor (e.g., cold sweating), increased salivation, sensations of bodily warmth, dizziness, drowsiness, headache, loss of appetite, and increased sensitivity to odors. Rapid and uncontrollable eye movements may correlate motion and vision imbalance. In dogs, signs of motion sickness include hyper-salivation (e.g., drooling), panting, swallowing, and lip-licking. As with humans, abdominal heaving, retching, and vomiting may occur progressively. The adaptive display system may be communicatively connected with passenger monitoring devices. A monitoring device may be a camera to capture the external signs and reactions of motion sickness. A monitoring device may be a wearable device to monitor internal signs, such as heart rate. A monitoring device may be a microphone to detect signs such as panting or increased respiration. The adaptive display system may monitor these indicators of motion sickness in both humans and dogs.

If the adaptive display system receives indications, such as from passenger monitoring devices, that a passenger continues to suffer from motion sickness, other adjustments may be made. For example, adjusting the temperature is particularly helpful to both humans and dogs that suffer from motion sickness as motion sickness has been shown to disrupt temperature regulation. Additionally, adjusting the location of fans or air flow from fans may help to alleviate motion sickness. Music and positive verbal instructions may be helpful for reducing motion sickness.

Adults and children may be satisfied by watching a display showing a movie or television show, and thus their attention is focused on a display which is showing the modified images of the adaptive display system. However, small children and animals may not stay as focused. The adaptive display system may receive input from a passenger monitoring device that a small child or animal is losing focus on the displayed image or again experiencing the effects of motion sickness. The adaptive display system may change the content of the image or video displayed to the small child or animal. For example, the adaptive display system may store a collection of images of content which is known to be of interest to a dog, such as a ball, a bone, and a cat the dog likes to chase. The adaptive display system, when receiving input from a passenger monitoring device that the dog is losing focus on the modified image and may be experiencing motion sickness, may change the content of the image to keep the dog's focus. The adaptive display system may provide a mechanism for custom images or video to be loaded and utilized by the system, such that each passenger may have specific content catered to their individual interests (e.g., the dog's favorite chew toy, a toddler's favorite teddy bear).

Dogs may interact with on-screen graphics, as well as visually categorize certain stimuli. Research has been performed to find what kind of content may be of interest to dogs, colors dogs see best, and the distance displays should be placed from a dog to keep the dog's attention. Integrating this information with the adaptive display system may assist in keeping a dog's attention and properly countering the motion sickness.

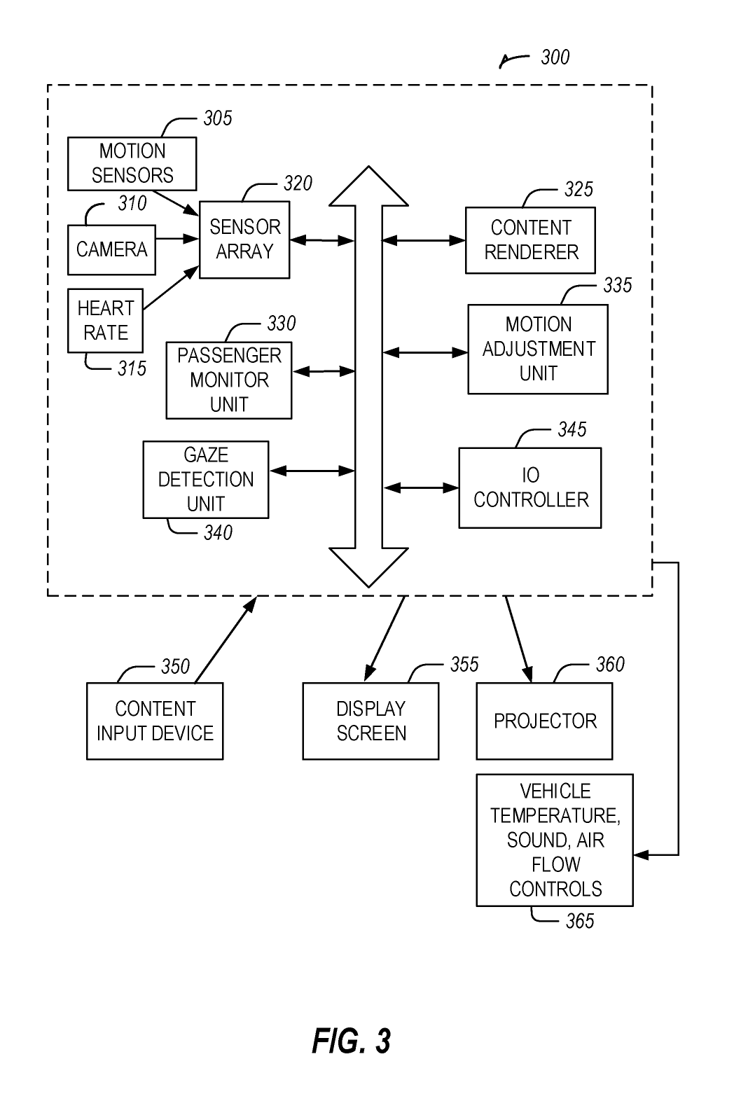

FIG. 3 illustrates the adaptive display engine 300 in accordance with some embodiments. In an embodiment, the adaptive display engine may receive visual content from a content input device 350. A content input device 350 may include a camera, a video game system, a video playback device (e.g., DVD player), and an image library (e.g., a collection of images specific to a young child or animal known to be of interest to them). The adaptive display engine 300 may receive visual content from a content input device 350 through the input/output (IO) controller 345. The IO controller 345 manages the content data received and the content data sent out for the adaptive display engine 300.

The adaptive display engine 350 includes a sensor array 320 to receive sensor input. The sensor array 320 may receive movement data from a set of motion sensors 305 attached to the vehicle. This may include accelerometers, gyroscopes, and GPS, to provide information about the physical movements the vehicle is experiencing. The sensor array 320 may send the data from the motion sensors 305 to the motion adjustment unit 335. The motion adjustment unit 335 may interpret the movement data of the vehicle into what the motion experience of a passenger may be. For example, when a vehicle makes a tight right turn, the vehicle leans to the left. However, the passenger may lean to the right in relation to the vehicle.

The motion adjustment unit 335 communicates the determined passenger motion experience to the content renderer 325. The content rendered 325 receives content data input from IO controller 345, such as a video. The content renderer 325 adjusts and modifiers the content data to move in correspondence with the movement sensed by the passenger. For example, if the passenger is sensing motion of leaning to the right, then the visual content may be adjusted to tilt to the right at a degree corresponding to the degree of tilt by the vehicle.

The content renderer 325 may communicate the modified visual content to the IO controller 345. The IO controller 345 may transmit the modified visual content to the presentation device being utilized in the vehicle, such as a display screen 355 or a projector 360.

The adaptive display engine 300 may include a gaze detection unit 340. The gaze detection unit 340 may request images from a camera 310, via the sensor array 320, of the passenger. The gaze detection unit 340 may analyze the images received from the camera 310 to determine the direction of the gaze of the passenger. This information may then be sent to the content renderer 325 to further adjust the visual content and how it is presented to a passenger. The content renderer 325 may determine how to adjust and modify the visual content based on the movements of the vehicle and determine the position to display the visual content such that it is in the direction of gaze of the passenger. A vehicle may have multiple display screens, such as one on that back of each front seat headrest, or a projector. The content renderer 325 may determine which screen is most closely within the direction of gaze of the passenger. The content rendered 325 may determine where to project the visual content with a projector that is most closely within the direction of gaze of the passenger.

The adaptive display engine 300 may continue to monitor the passenger for signs of continued motion sickness. The sensor array 320 may receive sensor data from a camera 310 or a wearable device to capture physiological data, such as a heart rate monitor 315. The camera 310 may capture images of the passenger to detect signs of motion sickness such as sweating and vomiting. The heart rate monitor 315 may capture an increased heart rate in the passenger. The data captured by a sensor is received by the sensor array 320 and sent to the passenger monitor unit 330. The passenger monitor unit 330 may utilize the sensor data to determine if the passenger continues to experience motion sickness. If the passenger still experiences motion sickness, the passenger monitor unit 330 may change other factors in the vehicle for the passenger to assist in alleviating motion sickness. For example, the passenger monitor unit 330 may adjust the controls 365 for the vehicle temperature, the sound directed at the passenger, or the air flow directed at the passenger.

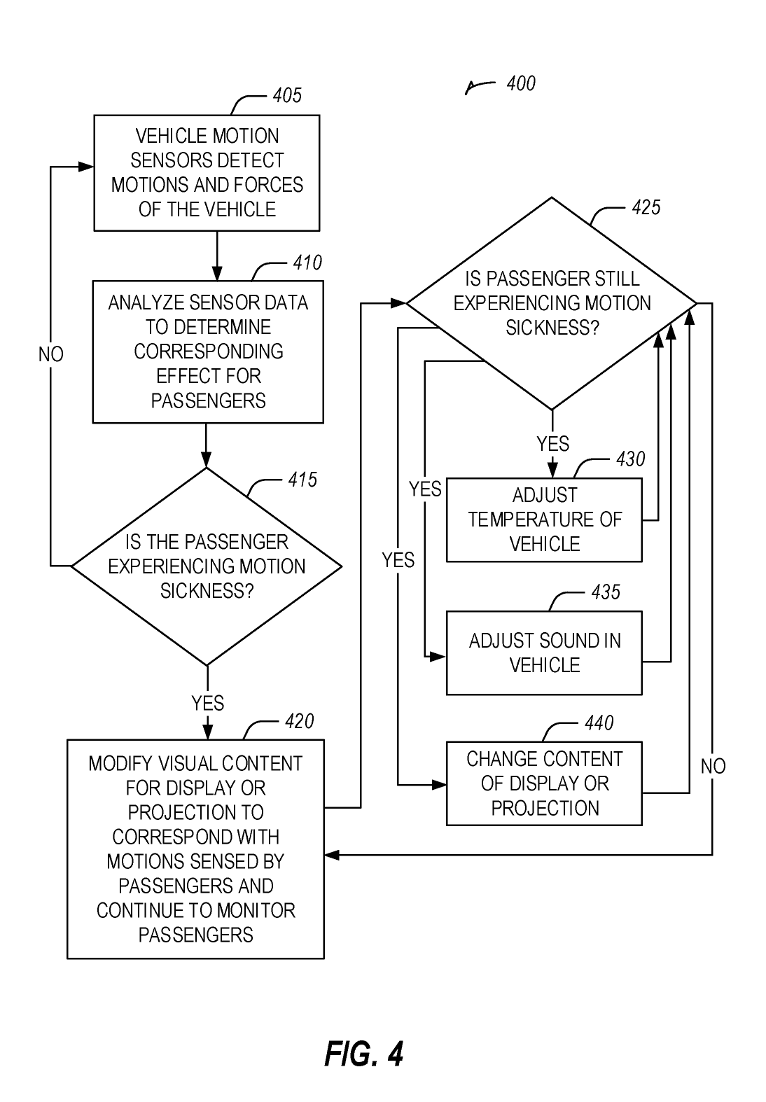

FIG. 4 illustrates a flow diagram 400 of an example of a process for adaptive display for preventing motion sickness, according to an embodiment. In an embodiment, the flow diagram 400 is a process for the adaptive display system to determine vehicle motion, modify visual content, and make further adjustments if the passenger is still experiencing motion sickness. At operation 405, the vehicle motion sensors detect the motions and forces of the vehicle. Sensors may include an accelerometer and a gyroscope to determine to provide data about the movement and motions of the vehicle body. At operation 410, the adaptive display system may analyze the sensor data and determine how the motions of the vehicle may be translated to motions sensed by the passengers. For example, if a vehicle takes a hard right turn, the system may determine how the motion is sensed to a passenger. At decision 415, the adaptive display system may determine if one or more passengers is experiencing motion sickness. The adaptive display system may utilize a camera or a wearable device to detect signs of motion sickness in a passenger. When the adaptive display system determines at decision 415 the passenger is not experiencing motion sickness, the adaptive display system will continue to perform operation 405, operation 410, and decision 415 until it is determined a passenger is experiencing motion sickness.

At decision 415, the adaptive display system may determine if the passenger is experiencing motion sickness and perform operation 420 to modify the visual content for display or projection for a passenger. Operation 420 may adjust and modify the shape, angle, position, and movement of the visual content to correspond with the motions sensed by the passenger. The adaptive display system may continue to monitor the passenger for continued signs of motions sickness while the visual content is modified.

At decision 425, the adaptive display system may determine if the passenger is still experiencing motion sickness. When the passenger is not experiencing motion sickness, the adaptive display system returns to operation 420 to continue modifying the visual content and monitoring the passenger for additional signs of motion sickness. As noted in decision 415, operation 420 may monitor a passenger through a passenger monitoring device, such as a camera or wearable device (e.g., smartwatch, fitness tracker, heart rate monitor).

At decision 425, the adaptive display may determine the passenger is still experiencing motion sickness and perform additional remedies. The adaptive display system may perform operation 430 to adjust the temperature of the vehicle for the passenger experiencing motion sickness. This may include changing the direction of air flow to the passenger. The adaptive display system may perform operation 435 to adjust the sound in the vehicle. Adjusting the sound may include changing the volume of audio being played. Adjusting the sound may include changing the content of the audio being played, such as playing soothing and relaxing music. It has also been shown that audio containing positive verbal instructions may assist a passenger dealing with motion sickness. The adaptive display system may perform operation 440 to change the content of the display or projection. The content may be changed to content the passenger finds soothing or enjoyable. In the case of the passenger being a small child or animal, the content may be changed to an item of interest.

As the adaptive display system monitors the passenger, such as at operation 420 for signs of motion sickness or signs of continued motion sickness after remedial actions have been taken, the adaptive display may record the remedial actions which have had a positive effect on the passenger. The adaptive display system may identify a passenger, such as a facial identification using a camera. The adaptive display system may create a profile for the passenger that is stored in a storage device connected to the adaptive display system. The adaptive displays system may monitor the passenger as the modifications to the visual content is applied to determine the types and degrees of modification that assist the passenger with motion sickness. For example, the visual content may zoom in and out as the vehicle accelerates and decelerates, however this modification may prove to have no remedial effects for the passenger. In another example, the adaptive display system may rotate the visual content based on the degree of rotational force experienced as a vehicle makes a turn. The adaptive display system, in monitoring the passenger, may determine the passenger receives the best effect by rotating the visual content half as much as the degree of rotation force experienced by the vehicle. These types of effective remedial action characteristics, along with actions such as adjusting the temperature of the vehicle, may be stored with the passenger profile in the storage device. When a passenger takes a subsequent trip in the vehicle, the adaptive display system may identify the passenger and access the passenger profile in the storage device. The adaptive display system may load the stored data for effective remedial actions for the passenger. The adaptive display system may perform the effective remedial actions and provide the passenger with a better experience as less trial and error is performed to find the remedial actions which are effective for the passenger.

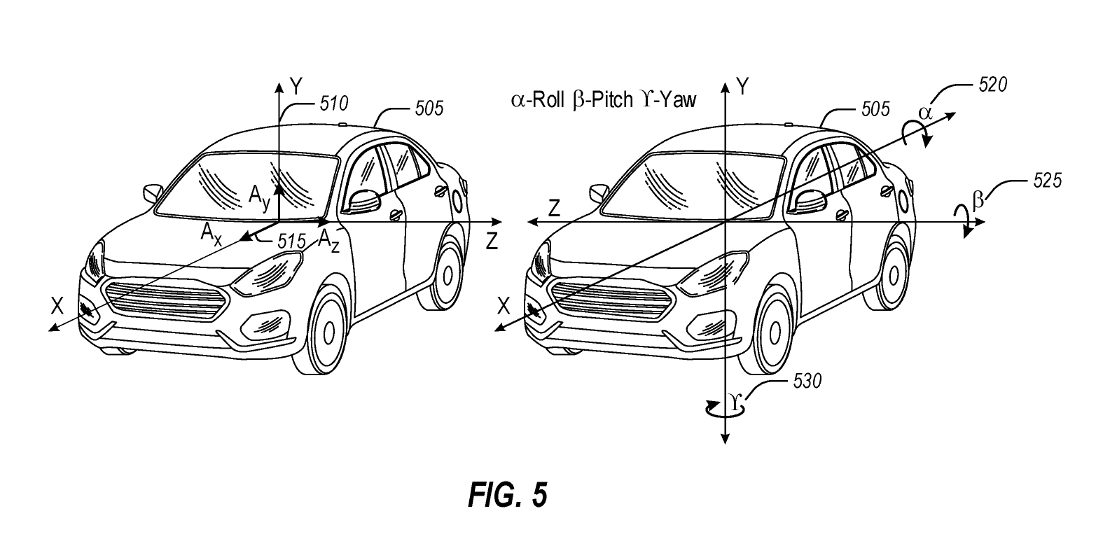

FIG. 5 illustrates the directions of movement a vehicle may experience, according to an embodiment. The vehicle 505 may move in three dimensions such as demonstrated by the Cartesian axis 510. The vehicle may move forward and backward along the X axis, side to side along the Z axis, and up and down along the Y axis, as well as any combination of the three. Acceleration or force in these directions may be represented by acceleration arrows 515.

A gyroscope sensor may provide measurements of the vehicles movement along a Cartesian axis (X, Y, Z) 510. An accelerometer may provide measurement of the vehicles acceleration or deceleration along the Cartesian axis (Ax, Ay, Az) 515. The gyroscope and other positioning sensors may provide information for changes in the vehicle's position in relation to the ground level, such as the roll 520 (α), the pitch 525 (β), and the yaw 530 (Υ).

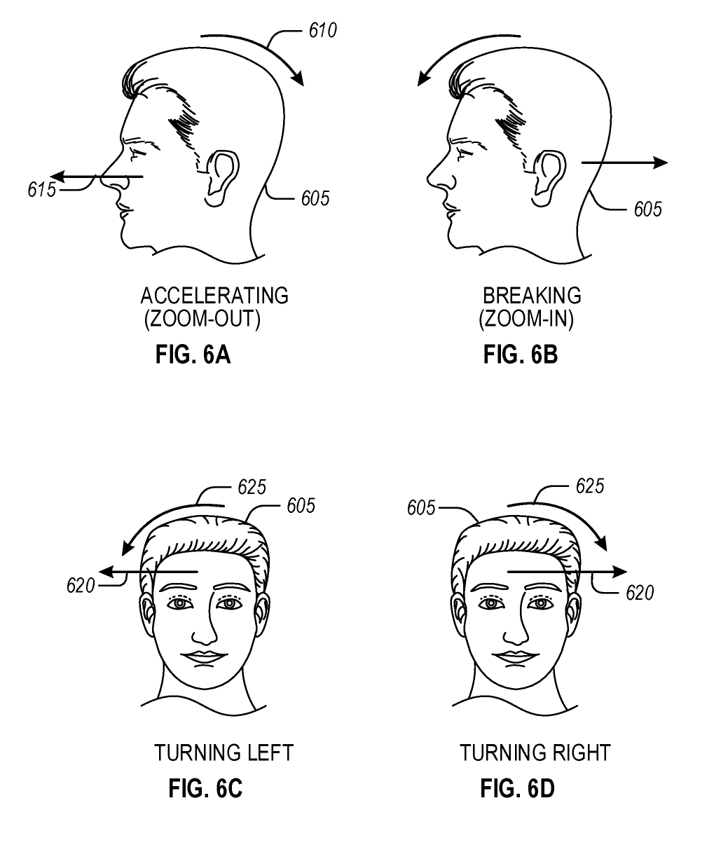

FIGS. 6A-6D illustrate examples of the motion forces a person may experience as a passenger in a vehicle in accordance with an embodiment.

The adaptive display system may modify the input image or video according to a set of rules corresponding to the motion effects on a passenger's head 605. In the following examples, roll, pitch, and yaw are applied in the opposite direction, such that Rα=−α, Rβ=−β, and RΥ=−Υ. For example, forward acceleration 615 (Ax) may trigger a Zoom-Out Effect (ZI) which may reflect minor increase of the image as a result of the head nod 610. This may also result in a pitch change as well (Rβ).

An example calculation for the amount the visual content should zoom in or out may be ZI=−Kz*Ax where ZI is floating number in percent specifying picture scale to be applied to the original picture in order to reflect horizontal acceleration along X axis. Kz is a constant value that is configurable per application and passenger preference.

An example calculation for the amount the visual content should shift upward because of the head not 610 may be VS=Kv*Ay where VS is floating number in percent specifying vertical shift of the picture along Y axis. Kv is a constant value that is configurable per application and passenger preference.

When a vehicle makes a turn, the passenger head may shift horizontally 620 and tilt 625 to one side or the other. An example calculation for the horizontal shift 620 may be HS=−Kh*Az where HS is floating number in percent specifying horizontal shift of the picture along Z axis. The horizontal shift (HS) may be augmented by head shake, thus Acceleration along the Z axis may be complemented by the picture rotation yaw (RΥ). Kh is a constant value that is configurable per application and passenger preference.

An example calculation for the picture rotation may be ZR=Kr*Az where ZR is a floating number measured in degrees and proportional to the acceleration. Kz is a constant value that is configurable per application and passenger preference.

The calculations may compensate vehicles roll 520 (α), pitch 525 (β), and yaw 530 (Υ) angles by rotating the picture in the opposite direction, such as when a passenger in a seated position is changing while external objects stay oriented as they were (e.g., trees remain standing vertical).

The above modifications may be performed by the adaptive display system which may calculate image size, distortion, and shift to modify original input visual content. The modifications to the visual content may be applied continuously such that the resulting displayed visual content may emulate a behavior as if the in the back seat was looking through the vehicle's windshield.

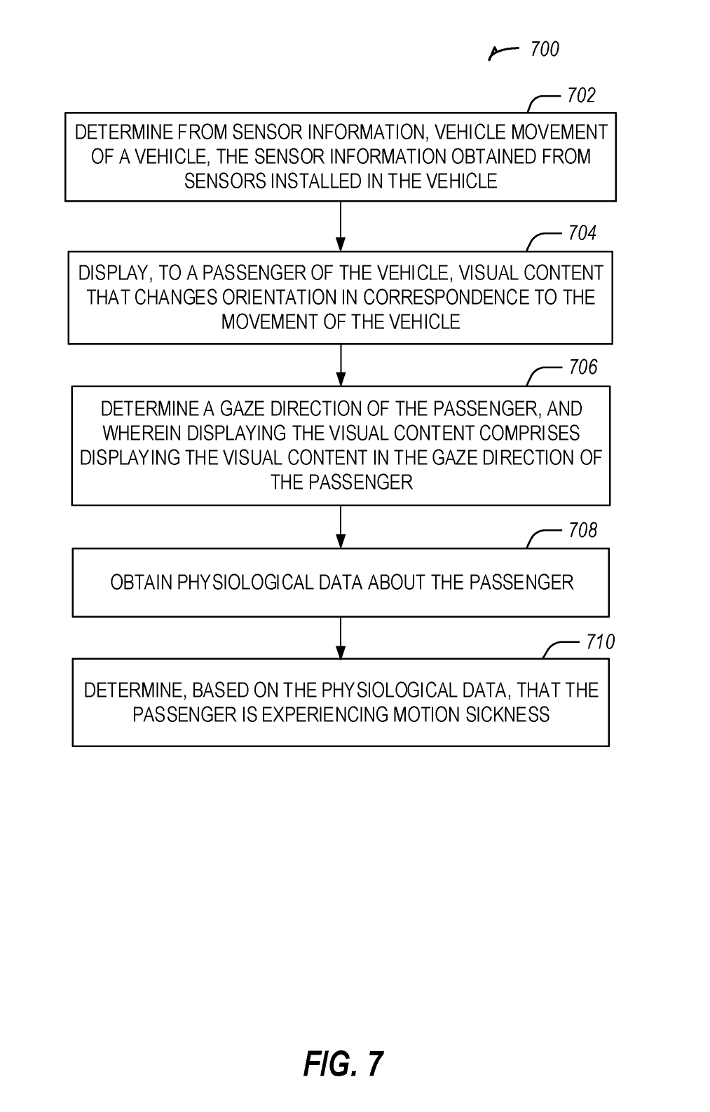

FIG. 7 illustrates a flowchart showing a technique 700 for adapting a display for motion sickness in a vehicle in accordance with some embodiments. The technique 700 includes an operation 702 to determine, from sensor information, vehicle movement of a vehicle, with the sensor information obtained from sensors installed in the vehicle. The sensors may include accelerometers and gyroscopes to describe the movements such as vehicle moving along an axis and pivoting on an axis. The technique 700 includes an operation 704 to display, to a passenger of the vehicle, visual content that changes orientation in correspondence to the movement of the vehicle. The visual content may move up and down or side to side as to correspond with the movement of the vehicle. The visual content may tilt to the left or to the right to correspond with the movement of the vehicle. The visual content may be zoomed in or zoomed out as the vehicle accelerates and decelerates. The display may be presented on a display screen, such as a flat panel display installed in the vehicle, or a through a projector. The visual content may be modified and moved to correspond to the movement of the vehicle, such as the visual content zooming in and out with acceleration and deceleration and tilting to the left or right as the vehicle tilts when making a turn. The visual content may be an image, a series of images, or a video. The visual content may include predetermined items of interest for the passenger, such as cartoon characters for a child. The vehicle may be one of an automobile, a bus, a train, a boat, or a plane.

The technique 700 may further include an operation 706 to determine a gaze direction of the passenger, and wherein displaying the visual content comprises displaying the visual content in the gaze direction of the passenger. The technique may include a camera to determine the direction a passenger is looking and then position the visual content within the passenger's field of view. This may be utilized for small children and animals that may not be inclined to turn their attention to a specific position. The technique may include projecting, with a projector, the visual content in the gaze direction of the passenger.

The technique 700 may further include an operation 708 to obtain physiological data about the passenger. The physiological data about a passenger may be obtained through wearable devices, such as a smartwatch or fitness tracker. The technique 700 may further include an operation 710 to determine, based on the physiological data, that the passenger is experiencing motion sickness. Signs of experiencing motion sickness may include vomiting, sweating, increased salivation, increase in body temperature, dizziness, drowsiness, headache, heavy breathing, and excessive swallowing.

Upon determining the passenger is experiencing motion sickness, the technique 700 may further include adjusting a temperature in the vehicle, adjusting a sound in the vehicle, adjusting an airflow directed at the passenger, and adjusting the content of the visual content. The technique 700 may include creating a passenger profile based on a visual identification from a camera, obtaining physiological data about the passenger, and determining, based on the physiological data, that the passenger is not experiencing motion sickness. This may indicate the adjustment steps taken were successful at remedying the motion sickness, thus the technique 700 may then store, in a storage device, the adjustment performed in association with the passenger profile. On a subsequent trip when the passenger returns to the vehicle, the technique 700 may further include identifying a passenger based on a visual identification from a camera and retrieving, from the storage device, an adjustment from the passenger profile associated with the identified passenger. The retrieved adjustment is performed, which may include at least one of content of the visual content, temperature of the vehicle, sound in the vehicle, or airflow to the passenger.

The technique 700 may further include identifying the passenger is an animal (e.g., a dog, a cat). The technique 700 may further include identifying the type of animal and identifying the color palette that type of animal may see most clearly. The visual content may be presented in the identified color palette. The type of animal may be identified with a camera or based on physiological data collected from a wearable device.

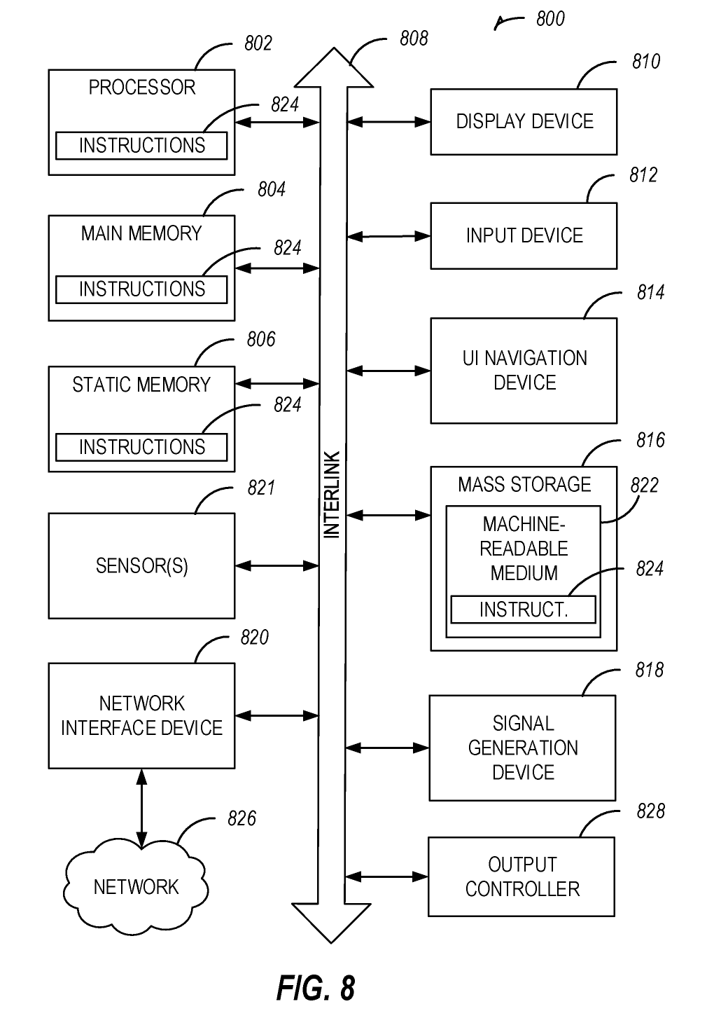

FIG. 8 illustrates a block diagram of an example machine 800 upon which any one or more of the techniques (e.g., methodologies) discussed herein may perform. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine readable medium 822 is illustrated as a single medium, the term “machine readable medium” may include a single medium or multiple media (e.g., a centralized or distributed database, and/or associated caches and servers) configured to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

### ADDITIONAL NOTES & EXAMPLES

Example 1 is a system for an adaptive display to prevent motion sickness comprising: at least one processor; and memory including instructions that, when executed by the at least one processor, cause the at least one processor to: determine from sensor information, movement of a vehicle, the sensor information obtained from a sensor installed in the vehicle; and display, to a passenger of the vehicle, visual content that changes orientation in correspondence to the movement of the vehicle.

In Example 2, the subject matter of Example 1 includes, instructions to: determine a gaze direction of the passenger, and wherein displaying the visual content comprises displaying the visual content in the gaze direction of the passenger.

In Example 3, the subject matter of Example 2 includes, wherein the visual content is projected to the direction of gaze of the passenger using a projector.

In Example 4, the subject matter of Examples 2-3 includes, wherein a display screen is selected to display the visual content based on the direction of gaze of the passenger.

In Example 5, the subject matter of Examples 1-4 includes, wherein the visual content is a video recording.

In Example 6, the subject matter of Examples 1-5 includes, instructions to: obtain physiological data about the passenger; and determine, based on the physiological data, that the passenger is experiencing motion sickness; and collect sensor information related to the movement of the vehicle.

In Example 7, the subject matter of Example 6 includes, instructions to: adjust a temperature in the vehicle, based on determining that the passenger is experiencing motion sickness.

In Example 8, the subject matter of Examples 6-7 includes, instructions to: adjust a sound in the vehicle, based on determining that the passenger is experiencing motion sickness.

In Example 9, the subject matter of Examples 6-8 includes, instructions to: adjust an airflow directed at the passenger, based on determining that the passenger is experiencing motion sickness.

In Example 10, the subject matter of Examples 6-9 includes, instructions to: select visual content for the passenger, based on determining the passenger is experiencing motion sickness.

In Example 11, the subject matter of Examples 6-10 includes, instructions to: perform an adjustment to at least one of: the visual content selected for the passenger, temperature of the vehicle, sound in the vehicle, or airflow to the passenger; create a passenger profile based on a first visual identification from a camera; obtain physiological data about the passenger; determine, based on the physiological data, the passenger is not experiencing motion sickness; and store, in a storage device, the adjustment associated with the passenger profile.

In Example 12, the subject matter of Example 11 includes, instructions to: identify the passenger based on a second visual identification from the camera; retrieve, from the storage device, adjustment data from the passenger profile corresponding to the passenger; and perform an adjustment, based on the adjustment data, to at least one of: the visual content selected for the passenger, temperature of the vehicle, sound in the vehicle, or airflow to the passenger.

In Example 13, the subject matter of Examples 1-12 includes, wherein the visual content selected for the passenger is based on predetermined items of interest for the passenger.

In Example 14, the subject matter of Examples 1-13 includes, wherein the vehicle is: an automobile, a bus, a train, a boat, or a plane.

In Example 15, the subject matter of Examples 1-14 includes, wherein the passenger is an animal.

In Example 16, the subject matter of Example 15 includes, instructions to: identify a type of animal associated with the passenger; identify a color palette corresponding with the type of animal; and present the visual content using the color palette.

In Example 17, the subject matter of Example 16 includes, wherein identifying the type of animal is performed by a visual identification with a camera.

In Example 18, the subject matter of Examples 16-17 includes, wherein identifying the type of animal is performed by a physiological identification with a wearable device.

In Example 19, the subject matter of Examples 1-18 includes, instructions to: capture sensor information from a sensor on the vehicle.

In Example 20, the subject matter of Example 19 includes, wherein the sensor is one of an accelerometer or a gyroscope.

Example 21 is a method for an adaptive display to prevent motion sickness comprising: determining from sensor information, movement of a vehicle, the sensor information obtained from a sensor installed in the vehicle; and displaying, to a passenger of the vehicle, visual content that changes orientation in correspondence to the movement of the vehicle.

In Example 22, the subject matter of Example 21 includes, determining a gaze direction of the passenger, and wherein displaying the visual content comprises displaying the visual content in the gaze direction of the passenger.

In Example 23, the subject matter of Example 22 includes, wherein the visual content is projected to the direction of gaze of the passenger using a projector.

In Example 24, the subject matter of Examples 22-23 includes, wherein a display screen is selected to display the visual content based on the direction of gaze of the passenger.

In Example 25, the subject matter of Examples 21-24 includes, wherein the visual content is a video recording.

In Example 26, the subject matter of Examples 21-25 includes, obtaining physiological data about the passenger; and determining, based on the physiological data, that the passenger is experiencing motion sickness; and collecting sensor information related to the movement of the vehicle.

In Example 27, the subject matter of Example 26 includes, adjusting a temperature in the vehicle, based on determining that the passenger is experiencing motion sickness.

In Example 28, the subject matter of Examples 26-27 includes, adjusting a sound in the vehicle, based on determining that the passenger is experiencing motion sickness.

In Example 29, the subject matter of Examples 26-28 includes, adjusting an airflow directed at the passenger, based on determining that the passenger is experiencing motion sickness.

In Example 30, the subject matter of Examples 26-29 includes, selecting visual content for the passenger, based on determining the passenger is experiencing motion sickness.

In Example 31, the subject matter of Examples 26-30 includes, performing an adjustment to at least one of: the visual content selected for the passenger, temperature of the vehicle, sound in the vehicle, or airflow to the passenger; creating a passenger profile based on a first visual identification from a camera; obtaining physiological data about the passenger; determining, based on the physiological data, the passenger is not experiencing motion sickness; and storing, in a storage device, the adjustment associated with the passenger profile.

In Example 32, the subject matter of Example 31 includes, identifying the passenger based on a second visual identification from the camera; retrieving, from the storage device, adjustment data from the passenger profile corresponding to the passenger; and performing an adjustment, based on the adjustment data, to at least one of: the visual content selected for the passenger, temperature of the vehicle, sound in the vehicle, or airflow to the passenger.

In Example 33, the subject matter of Examples 21-32 includes, wherein the visual content selected for the passenger is based on predetermined items of interest for the passenger.

In Example 34, the subject matter of Examples 21-33 includes, wherein the vehicle is: an automobile, a bus, a train, a boat, or a plane.

In Example 35, the subject matter of Examples 21-34 includes, wherein the passenger is an animal.

In Example 36, the subject matter of Example 35 includes, identifying a type of animal associated with the passenger; identifying a color palette corresponding with the type of animal; and presenting the visual content using the color palette.

In Example 37, the subject matter of Example 36 includes, wherein identifying the type of animal is performed by a visual identification with a camera.

In Example 38, the subject matter of Examples 36-37 includes, wherein identifying the type of animal is performed by a physiological identification with a wearable device.

In Example 39, the subject matter of Examples 21-38 includes, capturing sensor information from a sensor on the vehicle.

In Example 40, the subject matter of Example 39 includes, wherein the sensor is one of an accelerometer or a gyroscope.

Example 41 is at least one computer readable medium including instructions for an adaptive display to prevent motion sickness chunking that when executed by at least one processor, cause the at least one processor to: determine from sensor information, movement of a vehicle, the sensor information obtained from a sensor installed in the vehicle; and display, to a passenger of the vehicle, visual content that changes orientation in correspondence to the movement of the vehicle.

In Example 42, the subject matter of Example 41 includes, instructions to: determine a gaze direction of the passenger, and wherein displaying the visual content comprises displaying the visual content in the gaze direction of the passenger.

In Example 43, the subject matter of Example 42 includes, wherein the visual content is projected to the direction of gaze of the passenger using a projector.

In Example 44, the subject matter of Examples 42-43 includes, wherein a display screen is selected to display the visual content based on the direction of gaze of the passenger.

In Example 45, the subject matter of Examples 41-44 includes, wherein the visual content is a video recording.

In Example 46, the subject matter of Examples 41-45 includes, instructions to: obtain physiological data about the passenger; and determine, based on the physiological data, that the passenger is experiencing motion sickness; and collect sensor information related to the movement of the vehicle.

In Example 47, the subject matter of Example 46 includes, instructions to: adjust a temperature in the vehicle, based on determining that the passenger is experiencing motion sickness.

In Example 48, the subject matter of Examples 46-47 includes, instructions to: adjust a sound in the vehicle, based on determining that the passenger is experiencing motion sickness.

In Example 49, the subject matter of Examples 46-48 includes, instructions to: adjust an airflow directed at the passenger, based on determining that the passenger is experiencing motion sickness.

In Example 50, the subject matter of Examples 46-49 includes, instructions to: select visual content for the passenger, based on determining the passenger is experiencing motion sickness.

In Example 51, the subject matter of Examples 46-50 includes, instructions to: perform an adjustment to at least one of: the visual content selected for the passenger, temperature of the vehicle, sound in the vehicle, or airflow to the passenger; create a passenger profile based on a first visual identification from a camera; obtain physiological data about the passenger; determine, based on the physiological data, the passenger is not experiencing motion sickness; and store, in a storage device, the adjustment associated with the passenger profile.

In Example 52, the subject matter of Example 51 includes, instructions to: identify the passenger based on a second visual identification from the camera; retrieve, from the storage device, adjustment data from the passenger profile corresponding to the passenger; and perform an adjustment, based on the adjustment data, to at least one of: the visual content selected for the passenger, temperature of the vehicle, sound in the vehicle, or airflow to the passenger.

In Example 53, the subject matter of Examples 41-52 includes, wherein the visual content selected for the passenger is based on predetermined items of interest for the passenger.

In Example 54, the subject matter of Examples 41-53 includes, wherein the vehicle is: an automobile, a bus, a train, a boat, or a plane.

In Example 55, the subject matter of Examples 41-54 includes, wherein the passenger is an animal.

In Example 56, the subject matter of Example 55 includes, instructions to: identify a type of animal associated with the passenger; identify a color palette corresponding with the type of animal; and present the visual content using the color palette.

In Example 57, the subject matter of Example 56 includes, wherein identifying the type of animal is performed by a visual identification with a camera.

In Example 58, the subject matter of Examples 56-57 includes, wherein identifying the type of animal is performed by a physiological identification with a wearable device.

In Example 59, the subject matter of Examples 41-58 includes, instructions to: capture sensor information from a sensor on the vehicle.

In Example 60, the subject matter of Example 59 includes, wherein the sensor is one of an accelerometer or a gyroscope.

Example 61 is a system for an adaptive display to prevent motion sickness, the system comprising: means for determining from sensor information, movement of a vehicle, the sensor information obtained from a sensor installed in the vehicle; and means for displaying, to a passenger of the vehicle, visual content that changes orientation in correspondence to the movement of the vehicle.

In Example 62, the subject matter of Example 61 includes, means for determining a gaze direction of the passenger, and wherein displaying the visual content comprises displaying the visual content in the gaze direction of the passenger.

In Example 63, the subject matter of Example 62 includes, wherein the visual content is projected to the direction of gaze of the passenger using a projector.

In Example 64, the subject matter of Examples 62-63 includes, wherein a display screen is selected to display the visual content based on the direction of gaze of the passenger.

In Example 65, the subject matter of Examples 61-64 includes, wherein the visual content is a video recording.

In Example 66, the subject matter of Examples 61-65 includes, means for obtaining physiological data about the passenger; and means for determining, based on the physiological data, that the passenger is experiencing motion sickness; and means for collecting sensor information related to the movement of the vehicle.

In Example 67, the subject matter of Example 66 includes, means for adjusting a temperature in the vehicle, based on determining that the passenger is experiencing motion sickness.

In Example 68, the subject matter of Examples 66-67 includes, means for adjusting a sound in the vehicle, based on determining that the passenger is experiencing motion sickness.

In Example 69, the subject matter of Examples 66-68 includes, means for adjusting an airflow directed at the passenger, based on determining that the passenger is experiencing motion sickness.

In Example 70, the subject matter of Examples 66-69 includes, means for selecting visual content for the passenger, based on determining the passenger is experiencing motion sickness.

In Example 71, the subject matter of Examples 66-70 includes, means for performing an adjustment to at least one of: the visual content selected for the passenger, temperature of the vehicle, sound in the vehicle, or airflow to the passenger; means for creating a passenger profile based on a first visual identification from a camera; means for obtaining physiological data about the passenger; means for determining, based on the physiological data, the passenger is not experiencing motion sickness; and means for storing, in a storage device, the adjustment associated with the passenger profile.

In Example 72, the subject matter of Example 71 includes, means for identifying the passenger based on a second visual identification from the camera; means for retrieving, from the storage device, adjustment data from the passenger profile corresponding to the passenger; and means for performing an adjustment, based on the adjustment data, to at least one of: the visual content selected for the passenger, temperature of the vehicle, sound in the vehicle, or airflow to the passenger.

In Example 73, the subject matter of Examples 61-72 includes, wherein the visual content selected for the passenger is based on predetermined items of interest for the passenger.

In Example 74, the subject matter of Examples 61-73 includes, wherein the vehicle is: an automobile, a bus, a train, a boat, or a plane.

In Example 75, the subject matter of Examples 61-74 includes, wherein the passenger is an animal.

In Example 76, the subject matter of Example 75 includes, means for identifying a type of animal associated with the passenger; means for identifying a color palette corresponding with the type of animal; and means for presenting the visual content using the color palette.

In Example 77, the subject matter of Example 76 includes, wherein identifying the type of animal is performed by a visual identification with a camera.

In Example 78, the subject matter of Examples 76-77 includes, wherein identifying the type of animal is performed by a physiological identification with a wearable device.

In Example 79, the subject matter of Examples 61-78 includes, means for capturing sensor information from a sensor on the vehicle.

In Example 80, the subject matter of Example 79 includes, wherein the sensor is one of an accelerometer or a gyroscope.

Example 81 is at least one machine-readable medium including instructions that, when executed by processing circuitry, cause the processing circuitry to perform operations to implement of any of Examples 1-80.

Example 82 is an apparatus comprising means to implement of any of Examples 1-80.

Example 83 is a system to implement of any of Examples 1-80.

Example 84 is a method to implement of any of Examples 1-80.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A system for an adaptive display to prevent motion sickness comprising:
at least one processor; and
memory including instructions that, when executed by the at least one processor, cause the at least one processor to:
determine from sensor information, movement of a vehicle, the sensor information obtained from a sensor installed in the vehicle; and
display, to a passenger of the vehicle, visual content that changes orientation in correspondence to the movement of the vehicle.

2. The system of claim 1, further comprising instructions to:
determine a gaze direction of the passenger, and wherein displaying the visual content comprises displaying the visual content in the gaze direction of the passenger.

3. The system of claim 2, wherein the visual content is projected to the direction of gaze of the passenger using a projector.

4. The system of claim 1, further comprising instructions to:
obtain physiological data about the passenger; and
determine, based on the physiological data, that the passenger is experiencing motion sickness; and
collect sensor information related to the movement of the vehicle.

5. The system of claim 4, further comprising instructions to:
adjust a temperature in the vehicle, based on determining that the passenger is experiencing motion sickness.

6. The system of claim 4, further comprising instructions to:
adjust a sound in the vehicle, based on determining that the passenger is experiencing motion sickness.

7. The system of claim 4, further comprising instructions to:
adjust an airflow directed at the passenger, based on determining that the passenger is experiencing motion sickness.

8. The system of claim 4, further comprising instructions to:
select visual content for the passenger, based on determining the passenger is experiencing motion sickness.

9. A method for an adaptive display to prevent motion sickness comprising:
determining from sensor information, movement of a vehicle, the sensor information obtained from a sensor installed in the vehicle; and
displaying, to a passenger of the vehicle, visual content that changes orientation in correspondence to the movement of the vehicle.

10. The method of claim 9, further comprising:
determining a gaze direction of the passenger, and wherein displaying the visual content comprises displaying the visual content in the gaze direction of the passenger.

11. The method of claim 10, wherein the visual content is projected to the direction of gaze of the passenger using a projector.

12. The method of claim 9, further comprising:
obtaining physiological data about the passenger; and
determining, based on the physiological data, that the passenger is experiencing motion sickness; and
collecting sensor information related to the movement of the vehicle.

13. The method of claim 12, further comprising:
adjusting a temperature in the vehicle, based on determining that the passenger is experiencing motion sickness.

14. The method of claim 12, further comprising:
adjusting a sound in the vehicle, based on determining that the passenger is experiencing motion sickness.

15. The method of claim 12, further comprising:
adjusting an airflow directed at the passenger, based on determining that the passenger is experiencing motion sickness.

16. The method of claim 12, further comprising:
selecting visual content for the passenger, based on determining the passenger is experiencing motion sickness.

17. At least one non-transitory computer readable medium including instructions for an adaptive display to prevent motion sickness chunking that when executed by at least one processor, cause the at least one processor to:
determine from sensor information, movement of a vehicle, the sensor information obtained from a sensor installed in the vehicle; and
display, to a passenger of the vehicle, visual content that changes orientation in correspondence to the movement of the vehicle.

18. The at least one computer readable medium of claim 17, further comprising instructions to:
determine a gaze direction of the passenger, and wherein displaying the visual content comprises displaying the visual content in the gaze direction of the passenger.

19. The at least one computer readable medium of claim 18, wherein the visual content is projected to the direction of gaze of the passenger using a projector.

20. The at least one computer readable medium of claim 17, further comprising instructions to:
obtain physiological data about the passenger; and
determine, based on the physiological data, that the passenger is experiencing motion sickness; and
collect sensor information related to the movement of the vehicle.

21. The at least one computer readable medium of claim 20, further comprising instructions to:
adjust a temperature in the vehicle, based on determining that the passenger is experiencing motion sickness.

22. The at least one computer readable medium of claim 20, further comprising instructions to:
adjust a sound in the vehicle, based on determining that the passenger is experiencing motion sickness.

23. The at least one computer readable medium of claim 20, further comprising instructions to:
adjust an airflow directed at the passenger, based on determining that the passenger is experiencing motion sickness.

24. The at least one computer readable medium of claim 20, further comprising instructions to:
select visual content for the passenger, based on determining the passenger is experiencing motion sickness.
